SEQUENCE LISTING (2 sequences)

SEQ ID NO: 1 - protein, 198 aa, ARTIFICIAL
  SYNTHETIC CONSTRUCT
GELGTATGSHNTVTKTIETHTDNIETNMDENLRIPVTAEVGSGYFKMTDVSFDSDTLGKIKIRNGKSDAQMKEEDADLVITPVEGRALEVTVGQNLTFEGTFKVWNNTSRKINITGMQMVPKINPSKAFVGSSNTSSFTPVSIDEDEVGTFVCGTTFGAPIAATAGGNLFDMYVHVTYSGTETELQTKLAEFELQLLF

SEQ ID NO: 2 - DNA, 594 bp, ARTIFICIAL
  SYNTHETIC CONSTRUCT
ggcgaattgggtaccgctactggatcccataatactgttactaagactattgaaactcatactgataatattgaaactaatatggatgagaacctcagaatccctgttactgctgaagttggatctggatacttcaagatgactgatgtgtctttcgattctgatactctcggaaagatcaagatcagaaacggaaagtctgatgctcagatgaaggaagaggatgctgatctcgttatcactcctgttgagggaagagctttggaagttactgtgggacaaaatcttactttcgagggaactttcaaagtgtggaacaacacttctagaaagatcaacatcactggaatgcaaatggtgcctaagatcaacccttctaaggctttcgttggttcttctaacacttcttctttcacacctgtgtctatcgatgaggatgaagtgggaactttcgtgtgtggaactactttcggtgctcctattgctgcaacagcaggtggaaacctcttcgatatgtacgtgcacgtgacttattctggaactgagactgagctgcagaccaagcttgccgaattcgagctccagcttttgttc

(19) United States
(12) Patent Application Publication
(10) Pub. No.: US 2014/0170181 A1
(43) Pub. Date: Jun. 19, 2014

(54) VACCINE AGAINST WHITE SPOT SYNDROME VIRUS

(71) Applicants: Phang Siew Moi, Kuala Lumpur (MY); Gan Sook Yee, Kuala Lumpur (MY); Ung Eng Huan, Sabah (MY); Rofina Yasmin Othman, Kuala Lumpur (MY); Awang Muhammad Sagaf Abu Bakar, Sabah (MY)

(72) Inventors: Phang Siew Moi, Kuala Lumpur (MY); Gan Sook Yee, Kuala Lumpur (MY); Ung Eng Huan, Sabah (MY); Rofina Yasmin Othman, Kuala Lumpur (MY); Awang Muhammad Sagaf Abu Bakar, Sabah (MY)

(21) Appl. No.: 13/884,798

(22) PCT Filed: Oct. 31, 2011
(86) PCT No.: PCT/MY2011/000231

§ 371 Date: Jul. 17, 2013

(30) Foreign Application Priority Data
Nov. 10, 2010 (MY) PI2010005261

(51) Int. Cl. A61K 39/12; A23K 1/00; A23K 1/18

(52) U.S. Cl. CPC A61K 39/12; A23K 1/188; A23K 1/009
USPC 424/186.1

(57) ABSTRACT

A vaccine against White Spot Syndrome Baculovirus Complex infection in penaeid shrimp comprises algae expressing peptides having at least 70% similarity of amino acid sequence Seq No. 1, wherein the vaccine is administered to the penaeid shrimp as food additive.

### FIELD OF INVENTION

The present invention relates to a vaccine developed for providing protection to penaeid shrimp or prawn against infection caused by White Spot Syndrome Baculovirus Complex. More specifically, the disclosed invention is administered to the penaeid shrimp via oral ingestion, preferably as food additives.

### BACKGROUND OF THE INVENTION

Prawn culture, particularly species of the Penaeidae family, is a booming business with world population of 1 million metric tons which contributes widely to both economic development and employment. Among 20 viruses causing prawn disease, the White Spot Syndrome Virus (WSSV) causes USD1 billion losses per annum and species preference changes worldwide. WSSV was first discovered in Southeast Asia in the year 1992 and is currently the most serious viral pathogen towards prawn worldwide. It can cause up to 100% mortality within 7 to 10 days in commercial shrimp farms. The extreme virulence of WSSV and its wide coverage of host range have made it difficult to prevent and inhibit spread of the virus. Effort has been put in to develop a vaccine or prophylactic composition to fight against WSSV.

For example U.S. Pat. No. 6,440,466 discloses a composition for treating WSSV which contains an effective amount of extract from the plants Lantana camera, Aegle marmelos, Ocimum sanctum, Mimosa pudica, Cynodon dactylon, Curcuma longa, and Allium sativum. To enhance the prophylactic effect, the disclosed composition may be incorporated with carrier, diluents or excipients.

Another U.S. Pat. No. 6,705,556 describes a composition capable of promoting tolerance towards WSSV infection in the shrimp and the composition contains inactivated WSSV. Similarly, Vlak et al. provides an antigenic composition to immunize shrimp against WSSV in U.S. Pat. No. 6,908,616. Further immunogenic compositions are disclosed in U.S. patent no. 2007059808, U.S. Pat. Nos. 732,547, and 7,749,506.

Though the abovementioned compositions containing antigenic peptides are effective against the WSSV virus, the administered composition may not be voluntarily ingested by the shrimps and the peptides are subjected to degradation in an exposed environment. Other prior application suggest injection of the immunogenic composition to the shrimp to initiate the immunization, while such approach is not practical in aqua farming considering the numbers of shrimp reared in the ponds. Hence, an improved immunogenic composition prompting willing ingestion in the targeted crustacean is much desired.

### SUMMARY OF THE INVENTION

The present invention aims to provide an immunogenic composition or vaccine to be administered to penaeid shrimp to promote immunogenic activities against white spot syndrome virus.

Another object of the present invention is to disclose an orally administered vaccine against WSSV in shrimps that the vaccine is naturally ingested by the shrimp without the need of force feeding.

Further object of the present invention is to offer a food additive providing necessary nutrient to the shrimps besides developing immunization against WSSV in the shrimps. Specifically, the disclosed vaccine is delivered through a biological agent comprising various nutrient to promote growth of the shrimps.

At least one of the preceding objects is met, in whole or in part, by the present invention, in which one of the embodiment of the present invention involves a vaccine against White Spot Syndrome Baculovirus Complex infection in penaeid shrimp comprising algae expressing peptides having at least 70% similarity of amino acid sequence Seq No. 1, wherein the vaccine is administered to the penaeid shrimp as food additive.

In another aspect, the peptide is encoded in deoxyribonucleic acids sequence of Seq No. 2 or any other sequence with at least 70% similarity of Seq No. 2. Preferably, the deoxyribonucleic acids sequence of Seq No. 2 is incorporated into an expression vector carried in the algae for expression of the mentioned peptides. More preferably, the vector is pSV-beta-galactosidase control vector.

In another aspect, the algae is a recombinant organism which the preferred species is Chlorella vulgaris.

### BRIEF DESCRIPTION OF THE DRAWINGS

FIG. 1 shows the peptide sequence Seq No. 1 which is capable of promoting immunity against WSSV infection in the penaeid shrimp upon administration;

FIG. 2 shows one of the possible deoxyribonucleic acid sequences, Seq No. 2, encoding for the peptide sequence Seq No. 1;

FIG. 3 is a gel picture showing genomic DNA extracted from transformed strains and untransformed strain of Chlorella UMACC 001, namely WSSV 1: DNA extracted from transformed Chlorella strain 1; WSSV 2: DNA extracted from transformed Chlorella strain 2; WSSV 3: DNA extracted from transformed Chlorella strain 3; and UT1: DNA extracted from untransformed Chlorella strain 1;

FIG. 4 shows results of the putative transformed strains (6^{th} generation) which were selected by PCR analysis using primers that amplified the WSSV gene where Lane 1: PCR analysis conducted with DNA extracted from transformed Chlorella strain 1, WSSV1; Lane 2: PCR analysis conducted with DNA extracted from transformed Chlorella strain 2, WSSV2; Lane 3: PCR analysis conducted with DNA extracted from transformed Chlorella strain 3, WSSV3; Lane 4: PCR analysis conducted with DNA extracted from untransformed Chlorella strain 1, UT1; Lane 5: 1 kb DNA marker; Lane 6: PCR analysis conducted without DNA template (negative control); and Lane 7: PCR analysis conducted with the plasmid construct VP28 (positive control):

FIG. 5 shows results of a PCR analysis conducted for DNA extracted from transformed Chlorella that Lane 1: 100 bp DNA marker Lane 2: PCR analysis without any DNA template (negative control); Lane 3: PCR analysis of plasmid VP28-positive control; Lane 4, 5, 6: PCR analysis of DNA extracted from WSSV samples at 90^{th} generation, namely WSSV1, WSSV2, WSSV3, respectively;

FIG. 6 shows result of Southern blot analysis conducted with the DNA extracted from transformed strain WSSV3 (6^{th} generation) and untransformed strain (UT1, 6^{th} generation) indicating DNA integration;

FIG. 7 are histograms showing results obtained from a live viral challenge experiment on prawn fed with transformed algae as an oral vaccine;

FIG. 8 shows the native sequence of the VP28 peptide.

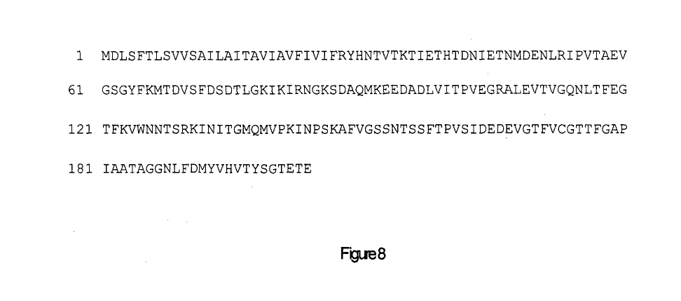

### DETAILED DESCRIPTION OF THE INVENTION

One skilled in the art will readily appreciate that the present invention is well adapted to carry out the objects and obtain the ends and advantages mentioned, as well as those inherent therein. The embodiment described herein is not intended as limitations on the scope of the invention.

The present invention includes a vaccine against White Spot Syndrome Baculovirus Complex infection in penaeid shrimp comprising algae expressing peptides having at least 70% similarity of amino acid sequence Seq No. 1, wherein the vaccine is administered to the penaeid shrimp as food additive. The amino acids sequence being expressed in the algae is antigenic peptides derived from the White Spot Syndrome Baculovirus Complex, particularly VP28 of the virus. Further, it is known in the art that mentioned VP28 peptides can be modified in such way to ease its expression or promote its antigenic property and so on. Thus, the expressed peptides in different embodiments of the present invention may be not totally identical to the original VP28 peptides found in the White Spot Syndrome Baculovirus Complex but rather at least 70% similarity as long the antigenic domains of the peptides are retained in the expressed peptides and the conformity of the domains is able to induce antigenic reaction in the penaeid shrimps.

In order to express the VP28 peptide and/or its derivatives, the mentioned algae of the present invention shall bear at least one copy of deoxyribonucleic acids (DNA) sequence of Seq No. 2 or any other deoxyribonucleic acids sequence of at least 70% similarity of Seq No. 2 which encodes for the amino acids sequence of the antigenic VP28. Likewise, one skilled in the art shall appreciate the fact that a single amino acid can be encoded by multiple deoxyribonucleic acids codon. Thus, the DNA template for expression of the VP28 in the present invention can be modified to achieve better expression rate, stability and so on, while still producing the preferred peptides with VP28 antigenic property. More preferably, the DNA template of the VP28 to be expressed is incorporated into a suitable vector. To facilitate the expression of the preferred peptides, pSV-beta-galactosidase control vector, but not limited to is employed in the present invention. The pSV-beta-galactosidase control vector used in the present invention also shows significant stability in the host cell that it is transferred from generation to generation along duplication and is capable of expressing the preferred antigenic peptides in the duplicated cells.

More specifically, the mentioned vaccine is preferably administered to the penaeid shrimp as routine food additives for long term exposure of the shrimp towards the antigenic peptides contained within the algae. It was found by the inventors of the present invention that continuous exposure to the antigenic peptides in the penaeid shrimp can increase resistance against this viral infection in the shrimp. It is believed the routine exposure allows development of active immunization against the infection.

Apart from the expression of the antigenic peptides, it is also crucial that the expressed peptides are readily ingested by the penaeid shrimps. As setting forth, ingestion of the expressed peptides alone containing the amino acid sequence Seq No. 1 into the penaeid shrimps is not voluntary. Moreover, administration of the antigenic peptides in the aqua farming environment may subject the expressed peptides to potential denaturation thus diminishing its antigenic property upon ingestion. While expressing the peptides in the algae, the disclosed invention ensures the antigenic peptides are ready to be ingested by the penaeid shrimps together with the algae as algae ingestion is natural behavior of the penaeid shrimps. The algae bearing copies of DNA template for expressing peptides having at least 70% similarity of amino acid sequence Seq No. 1 is a recombinant organism. Algae as the bio-factory platform of the VP28 antigenic peptides in the present invention can be mass-produced under specific conditions in the aqua farming environment to serve as a sustainable source of the antigenic peptides to the penaeid shrimps. Providing continuous exposure of the peptides having at least 70% similarity of amino acid sequence Seq No. 1 to penaeid shrimps to maintain the immunization against White Spot Syndrome Baculovirus Complex infection. Wrapped within cytoplasm of the algae, the produced antigenic peptides are shielded from potential denaturation caused by the water phase in the aqua farm especially fluctuation of pH in water. Besides, algae contains various nutrients such as essential amino acids, vitamin B12, beta-carotene, calcium, iron and so on. Thus, ingesting the algae carrying the antigenic peptides not only initiates the needed immunization but also promotes growth and health of the penaeid shrimps. More preferably, the algae employed for carrying the recombinant DNA and peptides is, but not limited to, Chlorella vulgaris. Other algae types may be employed in the present invention as well using different optimized parameters to insert the DNA template together with the vector into the host cells. The algae can be of freshwater or marine origin.

Preferably, the vaccine of the present invention is a prophylactic vaccine which is routinely fed to the penaeid shrimps to boost immunization against White Spot Syndrome Baculovirus Complex infection. Through the continuous exposure, the immunized penaeid shrimps show resistance towards infection of White Spot Syndrome virus. Still, in another embodiment, the vaccine is a prophylactic vaccine where it is administered to the infected penaeid shrimps to reduce the mortality rate and increase resistance against the disease.

The following examples are intended to further illustrate the invention, without any intent for the invention to be limited to the specific embodiments described therein.

### Example 1

Chlorella vulgaris UMACC 001 culture was obtained from the University of Malaya Algae Collection (UMACC). The microalga sample was cultured and maintained in the Algae Research Laboratory, Institute of Graduate Studies, University of Malaya. C. vulgaris was cultured in Bold's Basal Medium (BBM) (Nichols and Bold, 1965) at 25° C. and 282.45 μmol·s⁻¹ m⁻² (Phang and Chu, 2004).

### Example 2

The synthetic gene VP28 was assembled from synthetic oligonucleotides and cloned into plasmid pGA4 (ampR). The plasmid DNA was purified from transformed bacteria and concentration was determined by UV spectroscopy. The target gene was cloned into the BamHI and PstI site of pSVβ-gal to construct the pSV40WSSV vector. Transformation was carried out in E. coli Top 10 using the calcium chloride heat-shock method. This vector carries an ampicillin resistant marker and cell selection can be done using blue-white colony screening. The E. coli harboring the pSVβ-gal with the VP28 gene was further verified by restriction digestion and sequencing. The target gene was cloned into the lac Y region of pSVβ-gal thus creating a fusion peptide with the size of 42.5 kDa.

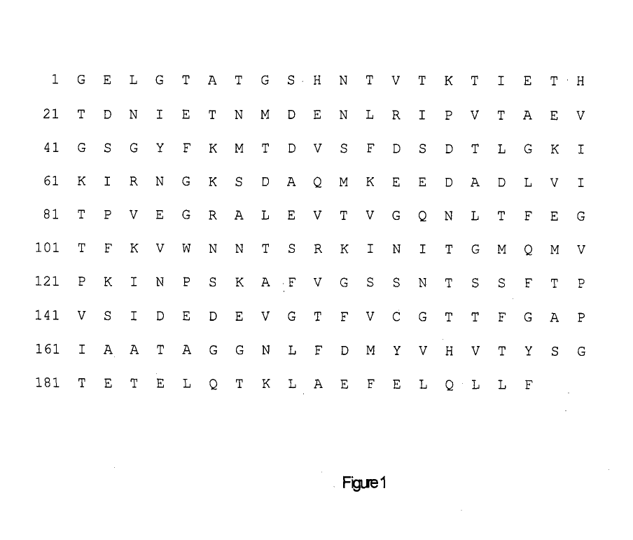

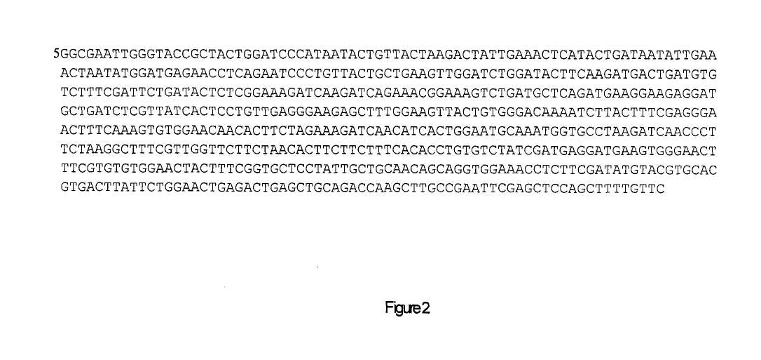

The partial VP28 gene was designed with nucleotide sequence optimized for expression. This gene is without the N-terminal hydrophobic region [Δ1-29] of the VP28 coat protein. The peptide region was designed based on Jeroen et al., 2004. The similarity index based on Martinez (1983)/Needleman and Wunsch (1970) DNA alignment is 68%. The translation map for native VP28 gene is shown in FIG. 1 while FIG. 2 shows the codon optimized VP28 gene.

### Example 3

The gold particles (Bio-Rad Laboratories. USA) sized 1.0 μm were encoated with the pSV40WSSV vector containing the VP28 gene. Fifty microlitres of gold particle solution (60 mg mL⁻¹) was mixed with 2 μL of a plasmid DNA solution (1 μL μg⁻¹), 50 μL of 2.5 M CaCl₂, and 20 μL of 0.1M spermidine. The mixture was vortexed and centrifuged to remove the supernatant. The remaining gold particles with plasmid DNA were resuspended in 250 μL 100% Ethanol and vortexed briefly for 10 s. Finally, 10 μL of gold-DNA particle was layered on a microcarrier for bombardment. C. vulgaris at a mid-log phase were bombarded using Bio-Rad PDS-1000/He Biolistic Particle Delivery System (Bio-Rad Laboratories, USA) at rupture disc pressure of 900 psi and at a distance of 9 cm. The bombarded and non-bombarded (control) C. vulgaris cultures were kept in BBM medium in the dark for two days before culturing into BBM agar plates.

### Example 4

Once the single colonies were visible as green colored clonal colonies, they were cultured in BBM medium separately until they reached exponential phase (OD₆₂₀ₙₘ=0.2), which was normally on the fourth day. One hundred millilitres of Chlorella vulgaris (OD₆₂₀ₙₘ=0.2) was harvested by centrifugation at 10,000 rpm for 10 min at room temperature. The total DNA was lysed in 550 μL lysis buffer (0.1M Tris-HCL, 0.05 M EDTA, 0.5 M NaCl and 1% BME) and homogenized by using a mortar and pestle for 3 min. Three microlitres of RNase A (10 mg mL⁻¹) and 35 μL of 20% SDS were added to the lysate and the microcentrifuge tube was inverted for five times before incubating at 65° C. for 1 hr. The protein was precipitated with 170 μL 5 M KAc and the microcentrifuge tube was inverted slowly for five times before incubating again in ice for 20 min. Then, 600 μL of chloroform:isoamyl (24:1) was added to eliminate the protein and the microcentrifuge tube was inverted for five times until the contents were well mixed. The mixture was centrifuged at 10,000 rpm at 4° C. for 10 min. The supernatant that contained the DNA was transferred into a clean microcentrifuge tube containing 500 μL of chilled isopropanol. The solution was gently mixed by inversion until thread-like strands of DNA formed a visible mass followed by centrifugation at 10,000 rpm for 10 min at 4° C. The supernatant was decanted and the pellet was washed with 500 μL of 70% ethanol at room temperature by gentle inversion. The DNA was recovered by centrifugation at 10,000 rpm for 5 min at 4° C. The ethanol was carefully aspirated by using a micropipette before inverting the tube onto clean absorbent paper and air-drying the pellet for 30 min. Then, the DNA was dissolved in 50 μL TE (pH 8.0) at 65° C. The DNA was stored at −20° C. until used.

The quantity and purity of the genomic DNA were determined by a biophotometer (Eppendorf, Germany) at OD₂₆₀ₙₘ and OD₂₈₀ₙₘ. The ratio between the absorbance values at 260 nm and 280 nm gave an estimate of the DNA purity. The quality and integrity of the DNA sample were also verified with 1.0% (w/v) agarose gel electrophoresis in 1×TAE buffer at 90V for 30 min. The genomic bands were viewed and photographed using AlphaImager™ 2200 (Alpha Innotech Corporation, USA).

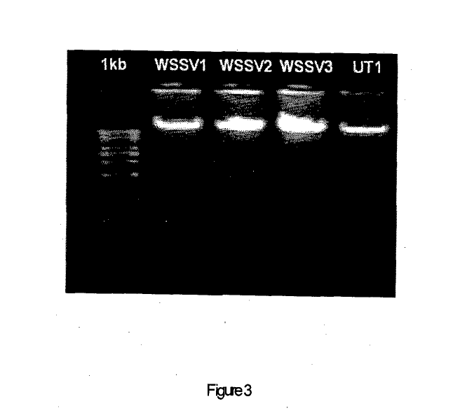

Genomic DNA was extracted from transformed strains and untransformed strain of Chlorella UMACC 001 when cultures were at 6^{th} generation (FIG. 3) based on specific growth rate of UMACC 001 as ranging from 0.22 to 0.30 per day. The purity of DNA obtained range from 1.80 to 2.00.

### Example 5

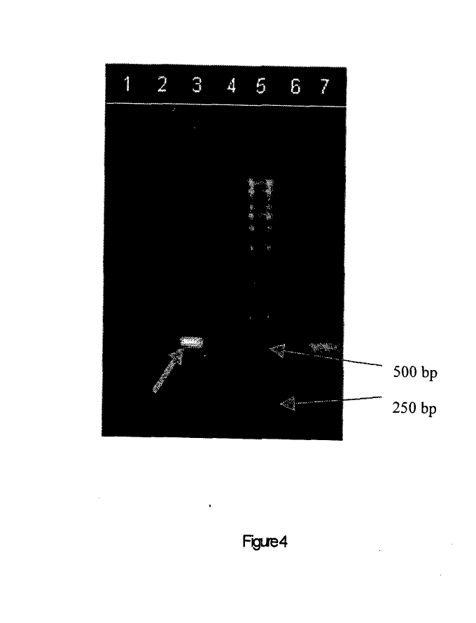

Two pairs of PCR primers were synthesized by Bio Basic Inc. (Malaysia). Partial VP28 gene fragment (573 bp) was amplified by specific primers: 5′-GCC GAA TTC GGA TCC CAT AAT ACT GTT AC-3′ and 5′-GCC AAG CTT CTC AGT CTC AGT TCC AGA AT-3′. The 25 μL PCR reaction consisted of 2.5 μL 10×PCR buffer, 0.5 μL MgCl₂ (100 mM), 0.4 μL dNTP mix (10 mM) (Bioron, Germany), 1 μL forward primer (10 μM), 1 μL reverse primer (10 μM), 2U Taq DNA Polymerase (Bioron, Germany), 1 μL genomic DNA (0.5 ng/μL) and 18.2 μL sterile deionized water. The PCR conditions were performed as follows: 5 min at 94° C. for pre-denaturation, 1 min at 94° C. to denature the double stranded DNA strand, 1 min at 55° C. to anneal the DNA and 2 min at 72° C. to extend the PCR amplified product. The denaturation, annealing and extension steps were repeated for 35 cycles. This was followed by a final extension at 72° C. for 10 min. The PCR products were analyzed with 1.0% (w/v) agarose gel electrophoresis in 1×TAE buffer at 90V for 30 min and viewed using AlphaImager™ 2200 (Alpha Innotech Corporation, USA). As shown in FIG. 4, putative transformed strains (colonies) were selected by PCR analysis using primers that amplified the WSSV gene (573 bp). Then, the PCR amplified bands were excised from the gel for DNA sequencing.

The DNA fragments from PCR were purified from the agarose gel using the QIAquick Gel Extraction Kit (Qiagen, Germany) according to supplier's protocol. Fifty microlitres of Buffer EB was applied to the column to elute the DNA and centrifuged at 10,000 rpm for 1 min at room temperature. Finally, the eluted DNA was sequenced using the same primers (5′-GCC GAA TTC GGA TCC CAT AAT ACT GTT AC-3′ and 5′-GCC AAG CTT CTC AGT CTC AGT TCC AGA AT-3′). The obtained sequence was compared with the sequence of VP28 which was incorporated into the construct to confirm that the amplified PCR fragment was the desired VP28 gene. Sequence analysis was conducted using ClustalW.

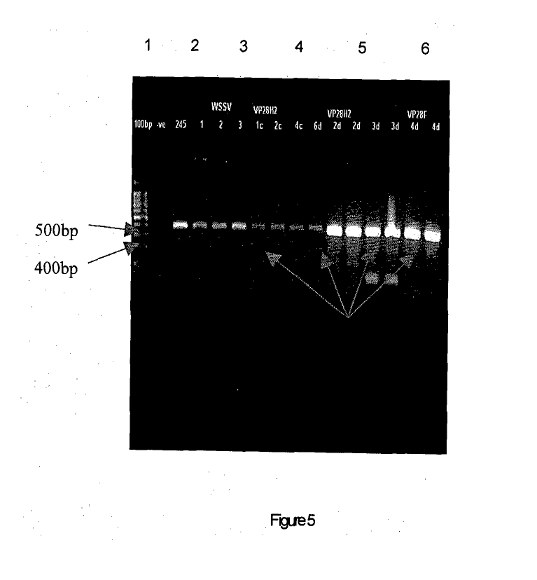

PCR analysis of partial VP28 gene fragment using primers set (5′-CCC TGT TAC TGC TGA AGT TGG-3′ and 5′-TGT TGC AGC AAT AGG AGC AC-3′) was also conducted for both transformed (WSSV1, WSSV2, and WSSV3) and non-transformed Chlorella harvested at 90^{th} generation (450 days after transformation) which generated a desired band of approximately 391 bp. The 25 μL PCR reaction consisted of 5.0 μL 5× GoTaq™ reaction buffer, 1.5 μL MgCl₂ (25 mM), 0.5 μL dNTP mix (10 mM), 0.5 μL forward primer (10 μM), 0.5 μL reverse primer (10 μM), 1U GoTaq™ DNA Polymerase (Promega. USA), 1 μL genomic DNA (1.0 μg/μL) and 15.8 μL sterile deionized water. The PCR conditions were performed as follows: 5 min at 94° C. for pre-denaturation, a total of 40 cycles of 1 min at 94° C. to denature the double stranded DNA strand, 1 min at 53° C. to anneal the DNA and 1 min at 72° C. to extend the PCR amplified product. FIG. 5 shows that the VP28 gene was still detected from the DNA extracted from culture of transformed Chlorella at 90^{th} generation.

### Example 6

Genomic DNA of the transformed positive clones (6^{th} cell generation) and untransformed DNA were digested for three days with restriction enzymes, BamHI and PstI (Promega, USA) which cut the VP 28 gene out from the construct, pSV40WSSV to give a band size of 573 bp. The digested products were separated by electrophoresing on 0.8% (w/v) agarose gel in 1×TAE at 90V for 30 min and viewed using AlphaImager™ 2200 (Alpha Innotech Corporation, USA).

After DNA fractionation, the gel was trimmed to remove unused areas of the gel. The DNA was depurinated in 0.2 M HCl for 30 min. Then, the DNA was denatured by soaking the gel in Denaturation Solution (1.5 M NaCl, 0.5 M NaOH) for 30 min with constant agitation. Then, the gel was briefly rinsed in deionized water, followed by soaking of the gel in Neutralization Buffer (0.5 M Tris, 1.5 M NaCl, pH 7.5) for 30 min with constant agitation. Meanwhile, charged nylon membrane, Hybond N+ (Amersham, U.K) was soaked in deionized water for 30 sec prior to soaking it in Nucleic Acid Transfer Buffer, 20×SSC (0.3M Tri-sodium citrate, 3 M NaCl) for 5 min.

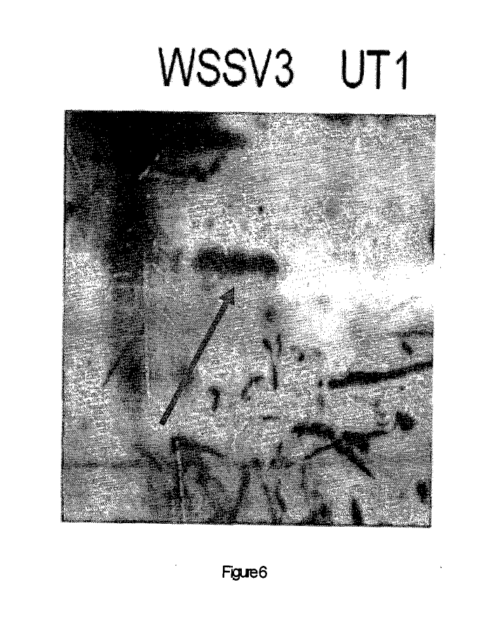

A 3 mm filter paper (Whatman, USA) was placed on a plastic platform in a blotting reservoir that was wider and longer than the gel. The ends of the filter paper were left to drape over the edges of the platform. The reservoir was filled with Nucleic Acid Transfer Buffer until the filter paper on the plastic platform became thoroughly wet, before smoothing out the air bubbles with a glass pipette. Then, the gel was removed from the solution and inverted so that its underside was at the uppermost and the gel was placed on the support. The top of the gel was moistened with Nucleic Acid Transfer Buffer so that the moistened membrane could be placed on the gel. Two pieces of 3 mm filter paper (Whatman, USA) were wet in Nucleic Acid Transfer Buffer and placed on the wet membrane avoiding any air bubble formation. Finally, paper towels (five to eight centimeters high) were cut and placed on the filter paper. A weight was put on the top of paper towel. The transfer of DNA was allowed for 24 hours. Then, the membrane was soaked in Neutralization Buffer for 20 min prior to hybridization. The target DNA was detected using the North2South Biotin Chemiluminescent Kit (Pierce Biotechnology, USA). The probe that was used for detection was the PCR product of pSV40WSSV amplified (573 bp) using the specific primers (as in reported in the PCR analysis). X-ray film was exposed to the membrane and a band (573 bp) was detected as expected. This indicated the integration of the WSSV gene into the host genome (FIG. 6).

### Example 7

The transformed Chlorella strains were scaled up from 10 ml tube cultures to 1 litre volume in magnetically stirred flasks placed in diffused sunlight as well as fluorescent tube lighting. This was then scaled up to a single 10 litre plastic cylindrical culture vessel before again subdividing into 4 such vessels giving a total culture of 40 litres. Algal growth was determined using a spectrophotometer as well as a haemocytometer. These were grown on a commercial algal growth media under the Epizyme® trademark following their instructions.

Once the culture vessels had reached a cell density of approximately 2×10⁷ cells/ml, they were harvested by pumping the entire volume using a DAB65 submersible pump connected to a Doulton ceramic filter. The concentrate was then stored in a refrigerator at 4° C. after cell numbers were once again determined using a haemocytometer. This was sonicated just before adsorption by dropping the concentrate onto commercial prawn pellets (Charoen Pokhphand Brand) and drying at 40° C. in a convection oven for 120 minutes.

For convenience, all samples prepared were standardized to 10⁸ cells/ml before application at a dosage of 150 ml/kg feed or 150 μl/g feed. This dosage was chosen as this corresponded to the maximum cell concentration possible used at the maximum practical adsorption rate without compromising the water stability of the feed pellet later in the water.

All prawns used were White Leg Shrimp Penaeus vannamei at an average of 8 g +/−0.5 g that had been pond-reared from SPF (specific pathogen free) post-larvae obtained from commercial hatcheries. After transfer in oxygenated containers from the ponds, these prawns were first acclimated to the BioSecure laboratory tanks for a period of 14 days. This was deemed necessary as these were clear acrylic tanks using clear seawater at 30 +/−0.5 ppt of salinity whereas the ponds had greenish high algal turbidity water and with a salinity of 28-32 ppt. During the first 14 days, they were fed with conventional prawn pellets. During the next 14 days, they were fed with the oral vaccine treated feed at the dosage mentioned earlier that is 10⁸ cells/ml at the rate of 150 μl/g pellet feed every single meal, 4 meals per day corresponding to approximately 3.5% of body weight. Finally, they were orally challenged by feeding frozen flesh from WSSV-PCR positive (bright band) prawns obtained from a recently WSSV-killed pond at approximately 6% of body weight. For the next 14 days post-oral virus challenge, they are fed oral vaccine treated feed (as in the first 2 weeks pre-oral challenge) and their moralities noted.

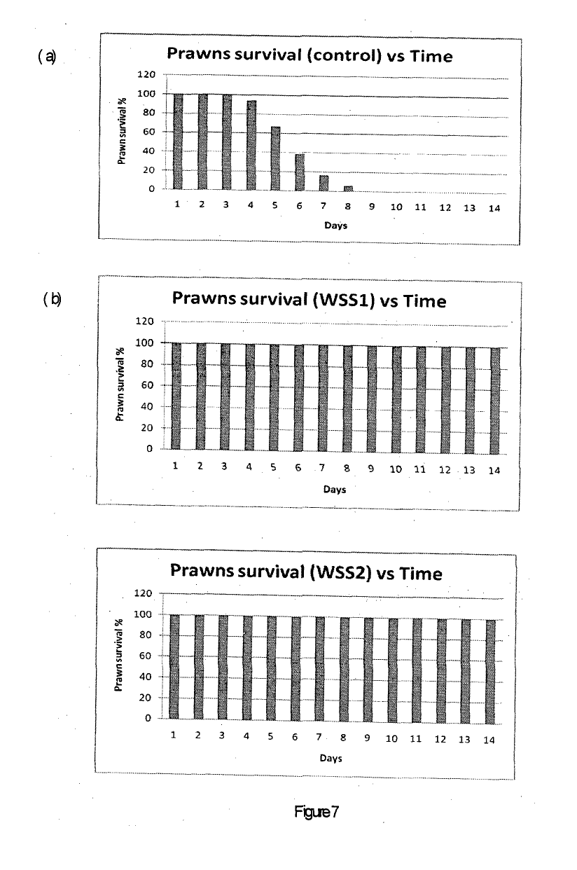

The transformed Chlorella, WSSV1 and WSSV2 showed positive results despite the 18 months duration between the time of transformation and the date of harvesting the algae and putting them in the refrigerator prior to challenge, there has not been any evidence that the gene was lost through lack of integration. It implies a very stable integration coupled with rapid growth as well as efficacy in challenge. The actual WSSV1 and WSSV2 challenge results are shown in FIG. 7.

In the untreated control, moralities began on day 3 post-challenge and by day 8, nearly all of the 20 prawns were dead. By day 9 post-challenge, indeed 100% mortality was observed in the control tank showing that the WSSV-infected carcass used was very much capable or causing 100% mortality within 9 days post-oral infection. The results clearly indicate that the clones WSSV1 and WSSV2 of the transformed VP28 Chlorella were successful in protecting the challenged prawns against acute death by WSSV.

The present disclosure includes as contained in the appended claims, as well as that of the foregoing description. Although this invention has been described in its preferred form with a degree of particularity, it is understood that the present disclosure of the preferred form has been made only by way of example and that numerous changes in the details of construction and the combination and arrangements of parts may be resorted to without departing from the scope of the invention.

The present disclosure includes as contained in the appended claims, as well as that of the foregoing description. Although this invention has been described in its preferred form with a degree of particularity, it is understood that the present disclosure of the preferred form has been made only by way of example and that numerous changes in the details of construction and the combination and arrangements of parts may be resorted to without departing from the scope of the invention.

### REFERENCE

1. Jeroen, W., Carolina, C. C., Just. M. V. and Mariëlle C. W. van Hulten. (2004). Protection of Penaeus monodon against White Spot Syndrom Virus by Oral Vaccination. Journal of Virology, 2057-2061.
2. Martinez, H. M. (1983). An Efficient Method for Finding DNA Repeats in Molecular Sequences. Nucleic Acids Research, 11: 4629-4634.
3. Needleman. S. B. and C. D. Wunsch (1970). A General Method Applicable to The Search for the Similarities in Amino Acid Sequence of Two Proteins. Journal of Molecular Biology, 48: 443-453.
4. Nichols, H. W. and Bold, H. C. (1965). Trichosarcina polymorpha gen. et sp. nov. Journal of Phycology 1: 34-38.
5. Phang, S. M. and Chu, W. L. (2004). The University of Malaya Algae Culture Collection (UMACC) and potential applications of a unique Chlorella from the collection. Japanese Journal of Phycology., 52: 221-224.

1-6. (canceled)

7. A vaccine against White Spot Syndrome Baculovirus Complex infection in penaeid shrimp, comprising:
a transformed recombinant algae expressing peptides having at least 70% similarity of amino acid sequence Seq No. 1, wherein the vaccine is administered to the penaeid shrimp as a food additive, wherein the algae is Chlorella vulgaris, wherein the peptide is expressed through a pSV-beta-galactosidase control vector incorporated with a deoxyribonucleic acid sequence of Seq No. 2 or any other sequence with at least 70% similarity of Seq No. 2 such that the deoxyribonucleic acid sequence is detectable in the transformed algae at a 90^{th} generation.

8. The vaccine of claim 1, wherein the vaccine is a prophylactic vaccine.
